(19) United States
(12) Patent Application Publication
(10) Pub. No.: US 2024/0398406 A1
(43) Pub. Date: Dec. 5, 2024

(54) HOLLOW HYDROGEL SUTURE FOR BIDIRECTIONAL LIQUID DELIVERY AND SENSING COMMUNICATION AND PREPARATION METHODS THEREOF

(71) Applicant: Army Medical University, Chongqing (CN)

(72) Inventors: Wen Zeng, Chongqing (CN); Fangchao Xue, Chongqing (CN); Shanlan Zhao, Chongqing (CN); Hao Tian, Chongqing (CN); Yanzhao Li, Chongqing (CN)

(73) Assignee: Army Medical University, Chongqing (CN)

(21) Appl. No.: 18/420,270

(22) Filed: Jan. 23, 2024

(30) Foreign Application Priority Data
May 30, 2023 (CN) 202310627598.6

(51) Int. Cl. A61B 17/06; A61B 17/00; A61L 17/10; A61M 5/172; B29C 41/00; B29C 41/08; B29C 41/22; B29C 41/42; B29D 22/00; B29K 29/00; B29K 105/00

(52) U.S. Cl. CPC A61B 17/06166; A61L 17/10; A61M 5/1723; B29C 41/003; B29C 41/08; B29C 41/22; B29C 41/42; B29D 22/00; A61B 2017/00526; A61B 2017/00893; A61B 2017/06185; A61M 2207/00; B29K 2029/04; B29K 2105/0061; B29K 2905/02; B29K 2995/0094; B29K 2995/0097

(57) ABSTRACT

Disclosed are a hollow hydrogel suture for bidirectional liquid delivery and sensing communication, and preparation methods thereof, which belong to the technical field of biomedicine. The hollow hydrogel suture includes a hydrogel suture body and a compound with a sensing communication function doped in the hydrogel suture body, wherein the compound with the sensing communication function is at least one of polypyrrole and copper-doped zinc sulfide, and the hydrogel suture body is made of polyvinyl alcohol.

### CROSS REFERENCE TO RELATED APPLICATION

This patent application claims the benefit and priority of Chinese Patent Application No. 2023106275986, entitled “Hollow hydrogel suture for bidirectional liquid delivery and sensing communication and preparation method thereof” filed on May 30, 2023, the disclosure of which is incorporated by reference herein in its entirety as part of the present application.

### TECHNICAL FIELD

The disclosure relates to the technical field of biomedicine, in particular to a hollow hydrogel suture for bidirectional liquid delivery and sensing communication, and preparation methods thereof.

### BACKGROUND ART

Organ damage and dysfunction are important factors leading to health deterioration and death. In many intervention measures, there still are challenges in timely detection and treatment of malignant events. The lack of simple and effective methods for diagnosing, treating, and monitoring damaged tissues, especially complex organs leads to doctors being unable to achieve real-time diagnosis and treatment of diseases. Tissue patches and gel that could load cells or drugs have shown great potential in the treatment of deep diseases in recent years. Existing tissue patches or gel suspensions could enter the body through surgery or injection, but these materials cannot accurately and closely adhere with the tissues. The adhesion of the patches will also increase the risk of tissue adhesion and limit the function of the tissues. On the other hand, tissue patches with preloaded drugs or cells could sustainably release cells or factors according to design, but drug release is usually non repetitive and non selective, and therapists cannot flexibly choose drugs based on disease progression. The monitoring of disease prognosis and the evaluation of treatment effectiveness usually rely on complex and expensive clinical instruments, and patient compliance and economic conditions impose huge challenges to disease monitoring and treatment, and there is a lag in disease monitoring and treatment.

Sensors that is able to monitor tissue lesions in real-time could maximize the advantages of treatment and provide timely feedback and intervention for adverse events after surgery or hospital discharge. At present, although flexible sensors have sensing functions, they lack the ability to integrate signal transduction and drug release. However, due to the density or hardness of conductive components in hard sensors, the efficiency of drug delivery is low, and hard sensors may negatively affect the normal physiological activities of tissues.

In contrast, surgical sutures, due to their broad clinical applications and close integration with tissues, provide a potential method for real-time diagnosis, evaluation, and treatment of organs and diseased tissues. The rapid development of hydrogels makes it possible to use bioelectronic sensing sutures. These sutures could achieve wireless sensing, drug elution, inhibition of bacterial growth by near-infrared photothermal conversion, and sensing functions. However, although these sutures could provide flexible treatment options, they are still limited by their single function in monitoring disease progression in deep tissues, and could not integrate multiple monitoring plans and provide flexible treatment.

### SUMMARY

In view of this, the present disclosure provides a hollow hydrogel suture for bidirectional liquid delivery and sensing communication and preparation methods thereof. The hollow hydrogel suture according to the present disclosure could effectively close a variety of tissues, transmit biological signals and deliver drugs, thus playing the integration role of diagnosis, treatment, and monitoring.

In order to achieve the above object, the present disclosure provides the following technical solutions.

Provided is a hollow hydrogel suture for bidirectional liquid delivery and sensing communication, including or consisting of a hydrogel suture body, and a compound with a sensing communication function, doped in the hydrogel suture body, wherein the compound with the sensing communication function is at least one selected from the group consisting of polypyrrole (PPy) and copper-doped zinc sulfide, and the hydrogel suture body is made of polyvinyl alcohol (PVA).

In some embodiments, a mass fraction of the compound with the sensing communication function in the hollow hydrogel suture ranges from 1% to 30%.

In some embodiments, the hollow hydrogel suture has an outer diameter of 80-1500 μm, an inner diameter of 50-1000 μm, and a length of 5-70 cm.

The present disclosure also provides a method for preparing the hollow hydrogel suture for bidirectional liquid delivery and sensing communication as described in the above technical solutions, which includes or consists of the following steps:

(1) mixing polyvinyl alcohol, the compound with the sensing communication function, and water, to obtain a mixed solution;

(2) applying the mixed solution onto a surface of a line shape mold, and drying, to form a coating on the surface of the line shape mold; and

(3) soaking the line shape mold with the surface covered by the coating in a sodium hydroxide solution, hydrating, and removing the line shape mold, to obtain the hollow hydrogel suture for bidirectional liquid delivery and sensing communication.

In some embodiments, based on a total mass of the compound with the sensing communication function and polyvinyl alcohol being 100%, a mass fraction of the polyvinyl alcohol is not less than 70%.

In some embodiments, the polyvinyl alcohol has a weight average molecular weight of 7.5-50 kD.

The present disclosure also provides another method for preparing the hollow hydrogel suture for bidirectional liquid delivery and sensing communication as described in above technical solutions, which includes or consists of the following steps:

(i) applying a first polyvinyl alcohol solution onto a surface of a line shape mold, and drying, to form a first polyvinyl alcohol coating on the surface of the line shape mold;

(ii) applying a mixed solution of polyvinyl alcohol and the compound with sensing communication function onto a surface of the first polyvinyl alcohol coating, and drying, to form a sensing coating on the surface of the first polyvinyl alcohol coating;

(iii) applying a second polyvinyl alcohol solution onto a surface of the sensing coating, and drying, to form a second polyvinyl alcohol coating, thereby to obtain the line shape mold coated with a composite coating; and

(iv) soaking the line shape mold coated with the composite coating in a sodium hydroxide solution, hydrating, and removing the line shape mold, to obtain the hollow hydrogel suture for bidirectional liquid delivery and sensing communication.

In some embodiments, the first polyvinyl alcohol solution in step (i) and the second polyvinyl alcohol solution in step (iii) each have a polyvinyl alcohol mass fraction of 10-50%.

In some embodiments, the applying and the drying in step (i) are performed for 1-5 times, and under the condition that the applying and the drying in step (i) are performed for more than once, the applying and the drying are alternately performed;

the applying and the drying in step (ii) are performed for 1-5 times, and under the condition that the applying and the drying in step (ii) are performed for more than once, the applying and the drying are alternately performed; and

the applying and the drying in step (iii) are performed for 1-5 times, and under the condition that the applying and the drying in step (iii) are performed for more than once, the applying and the drying are alternately performed.

In some embodiments, under the condition that the compound with sensing function includes both polypyrrole and copper-doped zinc sulfide, the step (ii) includes or is performed by

applying a mixed solution of polyvinyl alcohol and copper-doped zinc sulfide onto a surface of the first polyvinyl alcohol coating, and drying, to form a polyvinyl alcohol coating doped with copper-doped zinc sulfide on the surface of the first polyvinyl alcohol coating; applying a mixed solution of polyvinyl alcohol and polypyrrole onto a surface of the polyvinyl alcohol coating doped with copper-doped zinc sulfide, and drying, to form a polyvinyl alcohol coating doped with polypyrrole on the surface of the polyvinyl alcohol coating doped with copper-doped zinc sulfide; or applying a mixed solution of polyvinyl alcohol and polypyrrole onto a surface of the first polyvinyl alcohol coating, and drying, to form a polyvinyl alcohol coating doped with polypyrrole on the surface of the first polyvinyl alcohol coating; and applying a mixed solution of polyvinyl alcohol and copper-doped zinc sulfide onto a surface of the polyvinyl alcohol coating doped with polypyrrole, and drying, to form a polyvinyl alcohol coating doped with copper-doped zinc sulfide on the surface of the polyvinyl alcohol coating doped with polypyrrole.

The present disclosure provides a hollow hydrogel suture for bidirectional liquid delivery and sensing communication, which includes a hydrogel suture body, and a compound with a sensing communication function, doped in the hydrogel suture body, wherein the compound with the sensing communication function is at least one selected from the group consisting of polypyrrole and copper-doped zinc sulfide, and the hydrogel suture body is made of polyvinyl alcohol. In the present disclosure, at least one of polypyrrole and copper-doped zinc sulfide is doped in the hydrogel suture body, so that the hydrogel suture has at least one characteristic of conductivity and mechanoluminescence, and could transmit biological signals through the changes of resistance, potential and optical signals. In addition, the hydrogel suture according to the present disclosure has a hollow structure, which allows an injection pump to pump therapeutic drugs into the pathological tissue. The hollow hydrogel suture for bidirectional liquid delivery and sensing communication according to some embodiments of the present disclosure has the following beneficial effects:

(1) In this field, there are still enormous challenges in the diagnosis and treatment of deep injuries. The damage effects of organs or deep tissues are continuous, and the increasing inflammation and tissue necrosis continuously affect the repair and normal physiological state of the body, damaging human health. The complex clinical equipment and expensive treatment means limit the timely diagnosis and treatment of deep injuries and complications thereof. In the present disclosure, the most common surgical suture is used to construct a hollow hydrogel suture doped with at least one of polypyrrole and copper-doped zinc sulfide, which could transmit the information of the injured part in the form of electrical signals and chemical signals, and collect the electrophysiological signals, blood glucose, and inflammatory factor levels of the diseased part in real time, providing an intuitive and reliable basis for early diagnosis of diseases.

(2) How to administer drugs in a targeted and quantitative manner to promote inflammation resolution and tissue regeneration is an important challenge in the field. The hydrogel suture according to the present disclosure has a hollow structure, a micron channel, and matches with the interface of commonly used clinical syringes, which allows for targeted injection of drugs to the wound tissue through the micro channel, thereby achieving local controllable drug administration, reducing the level of wound inflammation, and promoting the in situ regeneration of tissue.

(3) The hollow hydrogel suture for bidirectional liquid delivery and sensing communication according to the present disclosure has the function of diagnosis-treatment-monitoring, could be used for the closure, monitoring and repair of damaged tissues, and could realize real-time collection and transmission of diverse electrical signals and biological signals, which is of great significance for the diagnosis of deep tissue injury and pathological state, and could enable doctors to evaluate and judge patients' health status in real time through portable devices.

### BRIEF DESCRIPTION OF THE DRAWINGS

FIG. 1 shows physical photographs of hydrogel suture bodies (PRIS) with different sizes.

FIGS. 2A to 2C show scanning electron microscope (SEM) images of a hollow hydrogel suture body (PRIS), in which, FIG. 2A shows an SEM image of PRIS suture surface; FIG. 2B shows an SEM image of PRIS suture pipeline ports at low magnification; and FIG. 2C shows an SEM image of PRIS suture pipeline ports at high magnification.

FIGS. 3A to 3C show SEM images of a hollow hydrogel suture for bidirectional liquid delivery and sensing communication (DTMS) according to an embodiment of the present disclosure, in which, FIG. 3A shows an SEM image of DTMS suture surface; FIG. 3B shows an SEM image of DTMS suture pipeline ports at low magnification; and FIG. 3C shows an SEM image of DTMS suture pipeline ports at high magnification.

FIG. 4 shows mechanical performance (stress-strain) of a hollow hydrogel suture for bidirectional liquid delivery and sensing communication (DTMS) according to an embodiment of the present disclosure.

FIGS. 5A and 5B show channel patency characterizations of a hollow hydrogel suture body (PRIS), in which, FIG. 5A shows fluorescein isothiocyanate (FITC) being perfused through PRIS's hollow pipes; and FIG. 5B shows FITC passing through the hollow pipe of PRIS and flowing out into a container.

FIGS. 6A and 6B show channel patency characterizations of a hollow hydrogel suture for bidirectional liquid delivery and sensing communication (DTMS) according to an embodiment of the present disclosure, in which, FIG. 6A shows FITC being perfused through DTMS's hollow pipes; and FIG. 6B shows FITC passing through the hollow pipe of DTMS and flowing out into a container.

FIG. 7 shows test results of a sensing performance (resistance change) of a hollow hydrogel suture for bidirectional liquid delivery and sensing communication (DTMS) according to an embodiment of the present disclosure.

FIG. 8 shows test results of sensing performance (voltage change) of a hollow hydrogel suture for bidirectional liquid delivery and sensing communication (DTMS) according to an embodiment of the present disclosure.

FIG. 9 shows result of muscle tissue sutured with a hollow hydrogel suture for bidirectional liquid delivery and sensing communication (DTMS) according to an embodiment of the present disclosure.

FIG. 10 shows results of transmitting electromyography signals when suturing muscle tissue with a hollow hydrogel suture for bidirectional liquid delivery and sensing communication (DTMS) according to an embodiment of the present disclosure.

FIG. 11 shows result of myocardial tissue sutured with a hollow hydrogel suture for bidirectional liquid delivery and sensing communication (DTMS) according to an embodiment of the present disclosure.

FIG. 12 shows results of transmitting electrocardiogram signals when suturing cardiac muscle tissue with a hollow hydrogel suture for bidirectional liquid delivery and sensing communication (DTMS) according to an embodiment of the present disclosure.

FIG. 13 shows results of near-infrared fluorescence imaging of drugs delivered on a myocardium surface of rats by a hollow hydrogel suture for bidirectional liquid delivery and sensing communication.

FIG. 14 shows histological staining results after improving myocardial infarction in rats with a hollow hydrogel suture for bidirectional liquid delivery and sensing communication.

FIG. 15 shows statistical analysis of histological staining results after improving myocardial infarction in rats with a hollow hydrogel suture for bidirectional liquid delivery and sensing communication.

FIG. 16 shows results of cardiac magnetic resonance examination after improving myocardial infarction of mini pigs by a hollow hydrogel suture for bidirectional liquid delivery and sensing communication according to an embodiment of the disclosure.

FIG. 17 shows a cross section of a mechanoluminescence hydrogel suture having an inner diameter of 80 μm, and an outer diameter of 100 μm.

### DETAILED DESCRIPTION OF THE EMBODIMENTS

The present disclosure provides a hollow hydrogel suture for bidirectional liquid delivery and sensing communication, including or consisting of a hydrogel suture body, and a compound with a sensing communication function, doped in the hydrogel suture body, wherein the compound with the sensing communication function is at least one selected from the group consisting of polypyrrole and copper-doped zinc sulfide, and the hydrogel suture body is made of polyvinyl alcohol.

In the present disclosure, polypyrrole has conductivity, and the introduction of polypyrrole into the hollow hydrogel suture could endow the hydrogel suture with good conductive function; while copper-doped zinc sulfide exhibits mechanoluminescence, and the introduction of copper-doped zinc sulfide into the hollow hydrogel suture could endow the hydrogel suture with the mechanoluminescence function. In specific embodiments of the present disclosure, according to actual needs, polypyrrole or copper-doped zinc sulfide or both of them could be introduced to the hydrogel suture. Under the condition that only polypyrrole is doped, the obtained hollow hydrogel suture is a conductive hydrogel suture; under the condition that only copper-doped zinc sulfide is doped, the obtained hollow hydrogel suture is a mechanoluminescence hydrogel suture; and under the condition that both polypyrrole and copper-doped zinc sulfide are doped, the obtained hollow hydrogel suture is a conductive and mechanoluminescence hydrogel suture. The hollow hydrogel suture according to the present disclosure has at least one function of conductivity and mechanoluminescence. After being connected with the sensor, the information of damaged tissues could be transmitted in the form of electrical and chemical signals, providing an intuitive and reliable basis for disease

diagnosis. In some embodiments of the present disclosure, a mass fraction of the compound with the sensing and communication function in the hollow hydrogel suture ranges from 1% to 30%. In some embodiments, under the condition that the compound with sensing and communication function is polypyrrole, a mass fraction of polypyrrole in the hollow hydrogel suture ranges from 1% to 20%, and preferably from 5% to 15%. In some embodiments, under the condition that the compound with sensing and communication function is copper-doped zinc sulfide, a mass fraction of copper-doped zinc sulfide in the hollow hydrogel suture ranges from 1% to 10%, and preferably from 3% to 8%. In some embodiments, under the condition that the compound with sensing and communication function includes both polypyrrole and copper-doped zinc sulfide, in the hollow hydrogel suture, a mass fraction of polypyrrole ranges from 1% to 20%, and a mass fraction of copper-doped zinc sulfide ranges from 1% to 10%.

In some embodiments of the present disclosure, the hollow hydrogel suture has an outer diameter of 80-1500 μm, preferably 80 μm, 100 μm, 150 μm, 300 μm, 500 μm, 1000 μm, or 1500 μm. In some embodiments, the hollow hydrogel suture has an inner diameter of 50-1000 μm, preferably 50 μm, 80 μm, 100 μm, 150 μm, 300 μm, 500 μm, or 1000 μm. In some embodiments, the hollow hydrogel suture has a length of 5-70 cm, and preferably 10-60 cm.

The present disclosure provides a method for preparing the hollow hydrogel suture for bidirectional liquid delivery and sensing communication as described in above technical solutions (Method I), including or consisting of the following steps:
(1) mixing polyvinyl alcohol, the compound with the sensing communication function, and water, to obtain a mixed solution;
(2) applying the mixed solution onto a surface of the line shape mold, and drying. to form a coating on the surface of the line shape mold; and
(3) soaking the line shape mold with the surface covered by the coating in a sodium hydroxide solution, hydrating, and removing the line shape mold, to obtain the hollow hydrogel suture for bidirectional liquid delivery and sensing communication.

In the present disclosure, polyvinyl alcohol, the compound with the sensing communication function, and water are mixed to obtain a mixed solution. In some embodiments of the present disclosure, the polyvinyl alcohol has a weight average molecular weight of 7.5-50 kD, and preferably 10-40 kD. In some embodiments, the polypyrrole has a molecular weight of 1-20 kD. In some embodiments, based on a total mass of the polyvinyl alcohol and the compound with a sensing communication function being 100%, a mass fraction of the polyvinyl alcohol is not less than 70%. In some embodiments, the water is deionized water. The mass fractions of polypyrrole and copper-doped zinc sulfide will not be repeated.

In some embodiments of the present disclosure, mixing polyvinyl alcohol, the compound with the sensing communication function, and water is performed by mixing polyvinyl alcohol and water, and stirring at a temperature of 60-100° C. for 12-24 hours while maintaining a pH value of 6-8 (preferably 7), to obtain a polyvinyl alcohol solution; adding the compound with the sensing communication function (i.e., polypyrrole and/or copper-doped zinc sulfide) to the polyvinyl alcohol solution, to obtain a mixed solution, wherein the polyvinyl alcohol solution has a polyvinyl alcohol concentration of 100-800 mg/mL.

After obtaining the mixed solution, the mixed solution is applied onto a surface of a line shape mold, and dried, to form a coating on the surface of the line shape mold. In the present disclosure, there is no special requirement for the material of the line shape mold, and materials well known to those skilled in the art, such as aluminum wire or glass wire, may be used. In some embodiments, the line shape mold is washed and dried before use. In the present disclosure, there is no special requirement for the applying means, and means well-known to those skilled in the art may be used. In specific embodiments of the present disclosure, a stepper motor is used to evenly pull the line shape mold, so that the line shape mold is pulled from the mixed solution, with the line shape mold being coated with a layer of the solution. In some embodiments, the line shape mold is pulled at a rate of 10 cm/min to 1 m/min. In specific embodiments of the present disclosure, the mixed solution is applied in an amount such that the mixed solution is uniformly retained on the surface of the line shape mold without forming condensation beads. In some embodiments of the present disclosure, the drying is performed by drying at room temperature or vacuum drying. In the present disclosure, there is no special requirement for drying conditions, as long as sufficient dry could be achieved.

In some embodiments of the present disclosure, the applying and the drying are performed for 1-5 times. Under the condition that the applying and the drying are performed for more than once, the applying and the drying are alternately performed. For example, under the condition that the applying and the drying are performed for five times, drying is performed after a first applying, followed by reapplying, redrying, which are repeated until 5 times of applying and 5 times of drying are completed.

After forming a coating on the surface of the line shape mold, the line shape mold with the coating is soaked in a sodium hydroxide solution and hydrated, and the line shape mold is then removed, to obtain the hollow hydrogel suture for bidirectional liquid delivery and sensing communication. In some embodiments of the present disclosure, the sodium hydroxide solution has a concentration of 4-8 mol/L, and preferably 6 mol/L. In some embodiments, the soaking is performed for 24-48 hours, and preferably 24 hours. In some embodiments, the soaking is performed at room temperature. In some embodiments, the hydrating is performed at room temperature. In some embodiments, the hydrating is performed for 48 hours. In some embodiments, the hydrating is performed by soaking or shake rinsing the line shape mold with a coating in deionized water after soaking in the sodium hydroxide solution.

During the soaking, hydroxyl groups of polyvinyl alcohol are deporotonated through the alkaline attack from OH ions, and the resulting O groups and free Na⁺ form a complex, which promotes the stretching and alignment of PVA chains, thereby forming a crystal area. After contacting with water, hydrogen bonds replace the PVA chain complexes, thus forming microcrystals. The formation of greater amount of microcrystals and stronger hydrogen bonds would result in the expelling of water molecules, thereby obtaining hydrogels with low swelling rate and high elasticity.

In the present disclosure, there is no special requirement for the means of removing the line shape mold, and the line shape mold may be directly pulled out.

The present disclosure also provides another method (Method II) for preparing the hollow hydrogel suture for bidirectional liquid delivery and sensing communication as described in above technical solutions, which includes or consists of the following steps:

(i) applying a first polyvinyl alcohol solution onto a surface of a line shape mold and drying, to form a first polyvinyl alcohol coating on the surface of the line shape mold;

(ii) applying a mixed solution of polyvinyl alcohol and polypyrrole or a mixed solution of polyvinyl alcohol and copper-doped zinc sulfide onto a surface of the first polyvinyl alcohol coating, and drying, to form a sensing coating on the surface of the first polyvinyl alcohol coating;

(iii) applying a second polyvinyl alcohol solution onto a surface of the sensing coating, and drying, to form a second polyvinyl alcohol coating, thereby to obtain the line shape mold coated with a composite coating; and

(iv) soaking the line shape mold coated with the composite coating in a sodium hydroxide solution, hydrating, and removing the line shape mold, to obtain the hollow hydrogel suture for bidirectional liquid delivery and sensing communication.

In the present disclosure, a first polyvinyl alcohol solution is applied onto a surface of the line shape mold, and dried, to form a first polyvinyl alcohol coating on the surface of the line shape mold. In some embodiments of the present disclosure, the first polyvinyl alcohol solution has a polyvinyl alcohol mass fraction of 10-50%, and preferably 10-20%. In some embodiments, the polyvinyl alcohol solution is prepared by a process including mixing polyvinyl alcohol with water, stirring at a temperature of 60-100° C. for 12-24 hours while maintaining a pH value of 6-8, to obtain a polyvinyl alcohol solution. In specific embodiments of the present disclosure, the polyvinyl alcohol solution is sealed and stored at room temperature.

In some embodiments of the present disclosure, the line shape mold is consistent with those described in the above technical solutions and will not be repeated here. In some embodiments, the line shape mold is washed and dried before use.

In some embodiments of the present disclosure, means for the applying is consistent with that described in the above technical solutions, and will not be repeated here. In some embodiments, the drying includes sequential drying at room temperature and drying in an oven. In some embodiments, the drying at room temperature is performed for 12 hours. In some embodiments, the drying in an oven is performed at 60° C. In some embodiments, the drying in an oven is performed for 12 hours. In some embodiments, the applying and the drying in step (i) are performed for 1-5 times, and preferably 5 times. Under the condition that the applying and the drying is performed more than once, the applying and the drying are alternately performed. The specific operations are the same as those described in above technical solutions, and will not be repeated here.

In the present disclosure, after forming the first polyvinyl alcohol coating, a mixed solution of polyvinyl alcohol and polypyrrole or a mixed solution of polyvinyl alcohol and copper-doped zinc sulfide is applied onto a surface of the first polyvinyl alcohol coating, and then dried, to form a sensing coating on the surface of the first polyvinyl alcohol coating. In some embodiments of present disclosure, in the mixed solution of polyvinyl alcohol and polypyrrole, based on a total mass of polyvinyl alcohol and polypyrrole being 100%, a mass fraction of polyvinyl alcohol ranges from 80% to 99%, and preferably from 85% to 90%; and a mass fraction of polypyrrole ranges from 1% to 20%, and preferably from 10% to 15%. In some embodiments, the mixed solution of the polyvinyl alcohol and polypyrrole is prepared by a process including or consisting of preparing a polyvinyl alcohol solution according to procedures described in the above technical solutions, and mixing the polyvinyl alcohol solution and polypyrrole, and heating while stirring a resulting mixture, to obtain the mixed solution of polyvinyl alcohol and polypyrrole (i.e., PVA-PPy solution). In some embodiments, the heating while stirring is performed at 90° C. In some embodiments, the heating while stirring is performed for 12 hours.

In some embodiments of the present disclosure, in the mixed solution of polyvinyl alcohol and copper-doped zinc sulfide (referred to as PVA-Cu:ZnS solution), based on a total mass of polyvinyl alcohol and copper-doped zinc sulfide being 100%, and a mass fraction of polyvinyl alcohol ranges from 90% to 99%, and preferably from 90% to 95%; and a mass fraction of copper-doped zinc sulfide ranges from 1% to 10%, and preferably 5% to 10%. In some embodiments, the mixed solution of polyvinyl alcohol and copper-doped zinc sulfide is prepared according to the same process for preparing the mixed solution of polyvinyl alcohol and polypyrrole, except for replacing polypyrrole with copper-doped zinc sulfide.

In some embodiments of the present disclosure, means for the applying and the drying in step (ii) are consistent with those described in step (i), and will not be repeated here. In some embosiments, the applying and the drying in step (ii) are performed for 1-5 times, and preferably 2 times. Under the condition that the applying and the drying are performed for more than once, the applying and the drying are alternately performed. In some embodiments, specific operations are the same as those described in above technical solutions, and will not be repeated here.

In the present disclosure, after forming a sensing coating, a second polyvinyl alcohol solution is applied onto a surface of the sensing coating and dried, to form a second polyvinyl alcohol coating, resulting in a line shape mold coated with a composite coating. In some embodiments of the present disclosure, the second polyvinyl alcohol solution used in step (iii) is the same as that used in step (i), and will not be repeated here. In some embodiments, means for the applying and the drying in step (iii) are the same as those used in step (i), and will not be repeated here. In some embodiments, the applying and the drying in step (iii) are performed for 1-5 times, and preferably 2. Under the condition that the applying and the drying are performed for more than once, the applying and the drying are alternately performed. In some embodiments, specific operations are the same as those described in above technical solutions, and will not be repeated here.

In the present disclosure, after obtaining the line shape mold coated with the composite coating, the line shape mold coated with the composite coating is soaked in a sodium hydroxide solution, and hydrated, and the line shape mold is then removed, to obtain the hollow hydrogel suture for bidirectional liquid delivery and sensing communication. In some embodiments of the present disclosure, the concentration of the sodium hydroxide solution, soaking conditions, and hydration conditions are the same as those defined in Method I, and will not be repeated here.

In some embodiments of the present disclosure, under the condition that the compound with sensing function includes or consists of both polypyrrole and copper-doped zinc sulfide, the step (ii) includes or is performed by applying a mixed solution of polyvinyl alcohol and copper-doped zinc sulfide onto a surface of the first polyvinyl alcohol coating, and drying, to form a polyvinyl alcohol coating doped with copper-doped zinc sulfide on the surface of the first polyvinyl alcohol coating; and applying a mixed solution of polyvinyl alcohol and polypyrrole onto a surface of the polyvinyl alcohol coating doped with copper-doped zinc sulfide, and drying, to form a polyvinyl alcohol coating doped with polypyrrole on the surface of the polyvinyl alcohol coating doped with copper-doped zinc sulfide; or

applying a mixed solution of polyvinyl alcohol and polypyrrole onto a surface of the first polyvinyl alcohol coating, and drying, to form a polyvinyl alcohol coating doped with polypyrrole on the surface of the first polyvinyl alcohol coating; and applying a mixed solution of polyvinyl alcohol and copper-doped zinc sulfide onto a surface of the polyvinyl alcohol coating doped with polypyrrole, and drying, to form a polyvinyl alcohol coating doped with copper-doped zinc sulfide on the surface of the polyvinyl alcohol coating doped with polypyrrole.

That is to say, after forming the first polyvinyl alcohol coating, a polyvinyl alcohol coating doped with polypyrrole may be prepared first, followed by preparing a polyvinyl alcohol coating doped with copper-doped zinc sulfide; or, a polyvinyl alcohol coating doped with copper-doped zinc sulfide may be prepared first, followed by preparing a polyvinyl alcohol coating doped with polypyrrole.

In some embodiments, during the preparation of the polyvinyl alcohol coating doped with polypyrrole and the polyvinyl alcohol coating doped with copper-doped zinc sulfide, the applying and the drying are performed for 1-5 times. In some embodiments, specific operations are the same as those described in above technical solutions, and will not be repeated here.

The present disclosure provides two methods for preparing the hollow hydrogel suture for bidirectional liquid delivery and sensing communication, and the product performance is similar. In Method I, the compound with sensing function is uniformly doped in polyvinyl alcohol hydrogel. In Method II, under the condition that the compound with sensing function is polypyrrole alone or copper-doped zinc sulfide alone, the hollow hydrogel suture consists of, from inner to outer, the first polyvinyl alcohol hydrogel layer, the polyvinyl alcohol hydrogel layer doped with polypyrrole (or copper-doped zinc sulfide), and the second polyvinyl alcohol hydrogel layer; under the condition that the compound with sensing function includes both polypyrrole and copper-doped zinc sulfide, the hollow hydrogel suture consists of, from inner to outer, the first polyvinyl alcohol hydrogel layer, the polyvinyl alcohol hydrogel layer doped with polypyrrole, the polyvinyl alcohol hydrogel layer doped with copper-doped zinc sulfide, and the second polyvinyl alcohol hydrogel layer, or consists of, from inner to outer, the first polyvinyl alcohol hydrogel layer, the polyvinyl alcohol hydrogel layer doped with copper-doped zinc sulfide, the polyvinyl alcohol hydrogel layer doped with polypyrrole, and the second polyvinyl alcohol hydrogel layer.

The hollow hydrogel suture according to the present disclosure has the functions of sensing communication and bidirectional liquid delivery. In some embodiments, during the use, one end of the hollow hydrogel suture could be connected to a needle of a syringe, the syringe being installed on a syringe pump, and a liquid therefore could be transmitted through the syringe pump. Alternatively, in some embodiments, a thread head of the hollow hydrogel suture according to the present disclosure is led out to the subcutaneous, and connected with a sensor, the sensor being configured to record the electrophysiological signal. In some embodiments, the sensor is an oscilloscope or a bluetooth electrocardiogram (ECG) module.

The technical solutions of the present disclosure will be clearly and completely described below in conjunction with examples. Obviously, the described examples are only a part of examples of the present disclosure, not all of them. Based on the examples in the present disclosure, all other examples obtained by ordinary technicians in the art without creative labor shall fall within the scope of the present disclosure.

### EXAMPLE 1

### Construction of a Hollow Hydrogel Suture for Bidirectional Liquid Delivery and Sensing Communication

(1) Preparation of a Hydrogel Suture Body:

PVA (from Alading, M_{w} of 20,500) was dissolved in deionized water at 90° C., and the resulting mixture was stirred at a constant speed until PVA was completely and evenly dissolved to obtain a transparent solution, i.e., a PVA solution with a concentration of 100 mg/mL.

Each of aluminum fiber molds (the aluminum fiber molds had an outer diameter of 50 μm, 80 μm, 100 μm, 250 μm, 350 μm, 950 μm, 1400 μm, respectively) was soaked in the PVA solution, pulled out from the PVA solution by a stepper motor at a constant speed, and dried overnight at room temperature. The soaking, pulling out, and drying were repeated 5 times, forming a PVA coating on the surface of each of the aluminum fiber molds.

Each of the aluminum fiber molds coated with the PVA coating was soaked in 6 mol/L sodium hydroxide solution for 24 h, then transferred into ultrapure water and soaked therein for 24 h, forming a hydrogel. Each of the aluminum fiber molds was gently removed from the hydrogel formed, obtaining a hollow hydrogel suture (i.e., the hydrogel suture body, labeled as PRIS).

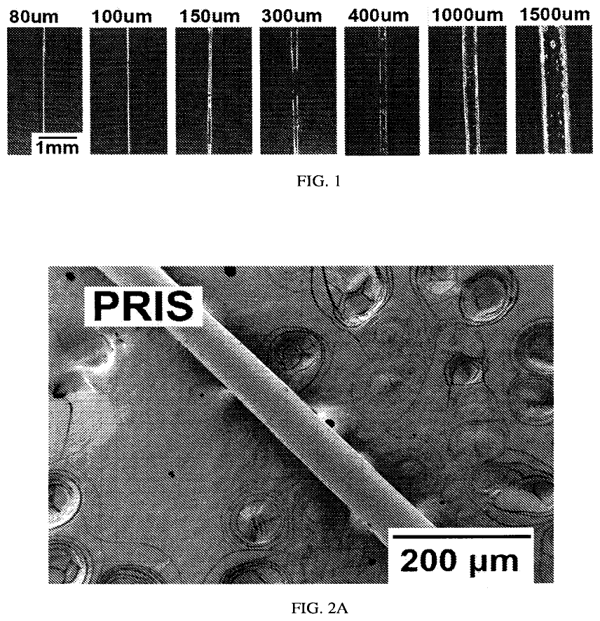

FIG. 1 shows physical photographs of hydrogel suture bodies (PRIS) with different sizes. A hollow hydrogel suture body (PRIS) having an outer diameter of 200 μm, and an inner diameter of 80 μm was also obtained.

### (2) Preparation of a Hollow Hydrogel Suture for Bidirectional Liquid Delivery and Sensing Communication (Conductive Hydrogel Suture):

The procedures are the same as the preparation of the hydrogel suture body, except that the PVA solution was replaced with a mixed solution of PVA and PPy, in which a mass ratio of PPy to PVA was 1:9, and the concentration of PVA was 800 mg/mL, obtaining a hollow hydrogel suture for bidirectional liquid delivery and sensing communication (labeled as DTMS).

A hollow hydrogel suture for bidirectional liquid delivery and sensing communication (DTMS) having an outer diameter of 200 μm, and an inner diameter of 80 μm according to an embodiment of the present disclosure was also obtained.

### (3) Scanning Electron Microscope Test

The test was performed as follows: the hydrogel suture was freeze-dried, and then adhered onto a surface of a conductive adhesive substrate for gold spraying treatment; the hydrogel suture body and the hydrogel suture for bidirectional liquid delivery and sensing communication after the gold spraying treatment were characterized by scanning electron microscope (from ZEISS).

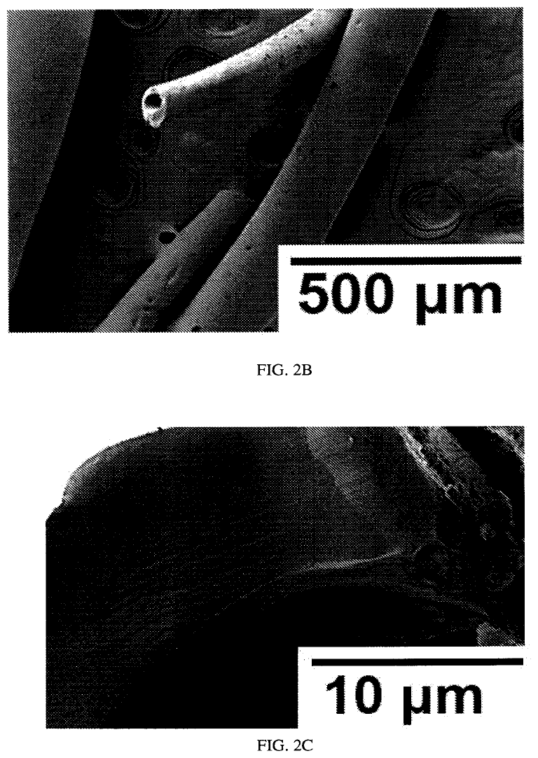

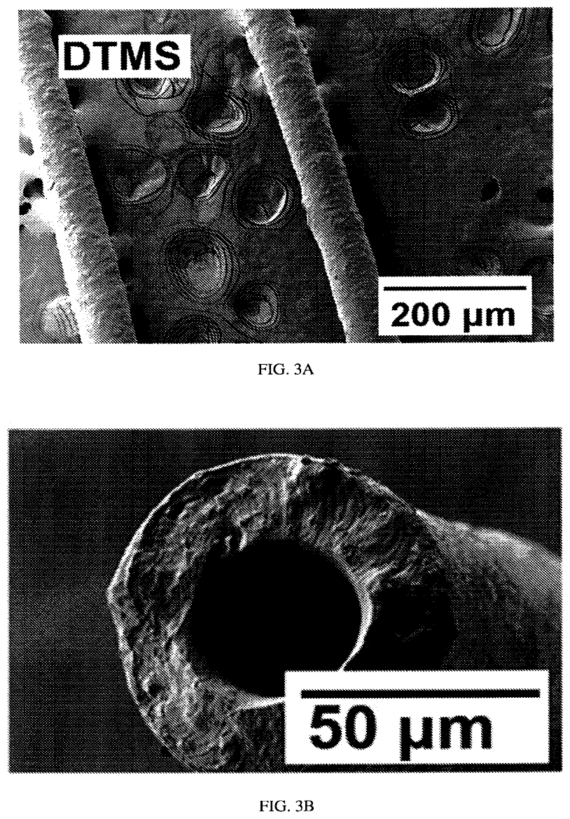

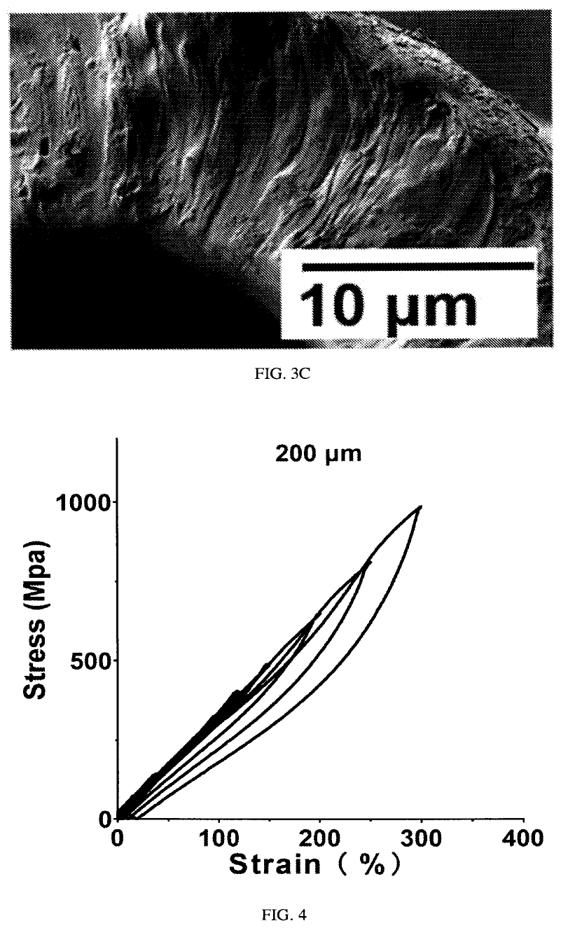

FIGS. 2A to 2C show SEM images of a hollow hydrogel suture body (PRIS) having an outer diameter of 100 μm and an inner diameter of 80 μm. FIGS. 3A to 3C show SEM images of a hollow hydrogel suture for bidirectional liquid delivery and sensing communication (DTMS) having an outer diameter of 80 μm and an inner diameter of 50 μm according to an embodiment of the present disclosure.

As can be seen from FIGS. 2A to 2C and FIGS. 3A to 3C, the hollow hydrogel suture body without any doping has a smooth surface, while there are fine particles on the surface of the hollow hydrogel suture doped with polypyrrole, which proves that the doping has been successfully achieved; the hydrogel suture has a micro channel, which could realize liquid delivery.

### (4) Mechanical Performance Tests

In order to test the mechanical performance of the hydrogel suture, a mechanical performance tester (QT-6201S) was used to test the stress-strain, breaking strength, tensile strength, and cyclic tensile performance. Specifically, a hydrogel suture having a length of 2 cm, an outer diameter of 200 μm, and an inner diameter of 100 μm was fixed on a fixture, and subjected to uniaxial stretch.

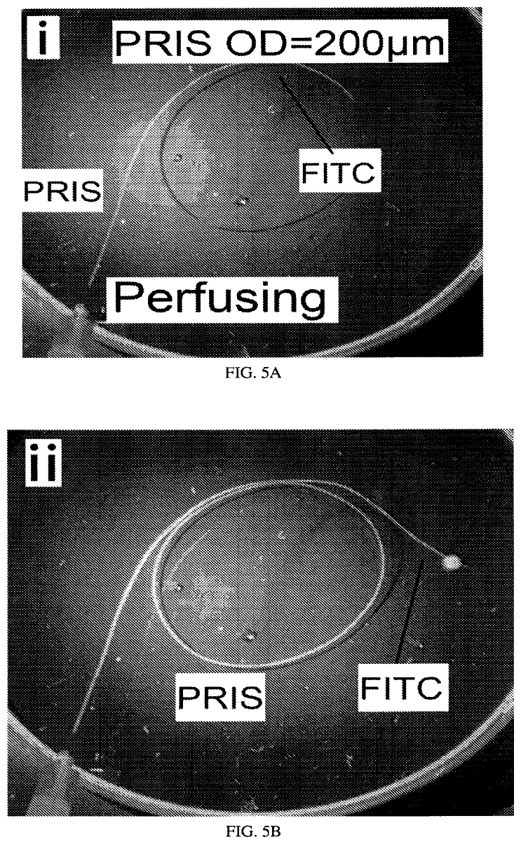

FIG. 4 shows test results of mechanical performance (stress-strain) of a hollow hydrogel suture for bidirectional liquid delivery and sensing communication (DTMS) according to an embodiment of the present disclosure. As can be seen from FIG. 4, the hydrogel suture has no obvious yield point within the deformation range of 20%-350%, and the energy dissipation is small, which proves that the hydrogel suture according to the present disclosure has good mechanical performance and elasticity.

### (5) Bidirectional Liquid Delivery Experiment

A hollow hydrogel suture body and a hollow hydrogel suture for bidirectional liquid delivery and sensing communication (i.e., conductive hydrogel suture) were prepared according to the procedures in step (1) and (2) respectively, each having an inner diameter of 80 μm and an outer diameter of 100 μm, and then subjected to a bidirectional liquid delivery experiment as follows:

The micro channel of the hydrogel suture was connected to a 34G syringe needle, which was connected to a syringe, the syringe being installed on a syringe pump. The syringe pump was started, and a fluorescein isothiocyanate (FITC) solution in the syringe pump flowed into a culture dish through the micro channel of the

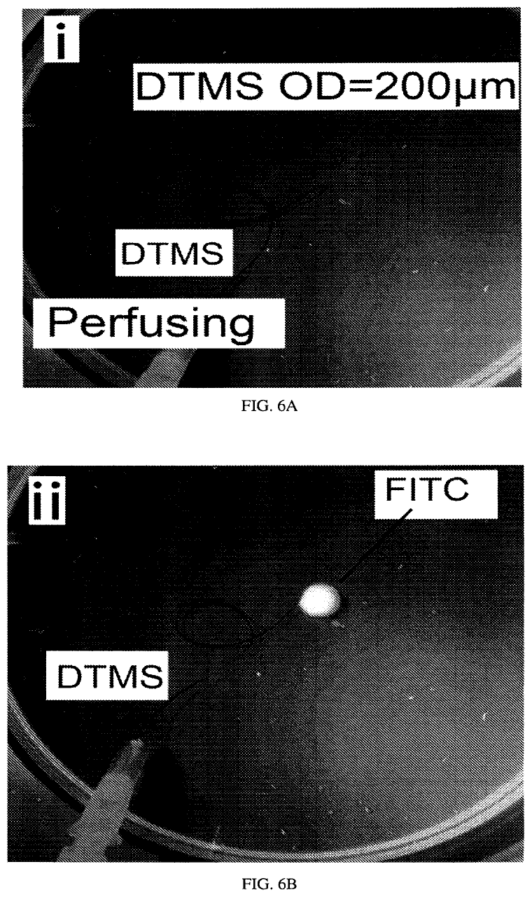

hydrogel suture. FIGS. 5A and 5B show channel patency characterizations of a hollow hydrogel suture body (PRIS). FIGS. 6A and 6B show channel patency characterizations of a hollow hydrogel suture for bidirectional liquid delivery and sensing communication (DTMS) according to an embodiment of the present disclosure.

As can be seen from FIGS. 5A to 5B and FIGS. 6A to 6B, both the hollow hydrogel suture body and the hollow hydrogel suture for bidirectional liquid delivery and sensing communication could realize bidirectional liquid delivery.

### (6) Motion Sensing Function Test

A hollow hydrogel suture for bidirectional liquid delivery and sensing communication was prepared according to the procedures in step (2), which had an inner diameter of 80 μm and an outer diameter of 100 μm, and then subjected to a motion sensing function test as follow:

The hollow hydrogel suture was fixed at the forefinger joint and the knee joint, and both ends of the hollow hydrogel suture were connected through wires and sealed and immobilized with polydimethylsiloxane (PDMS). The wires were connected to a resistance end pointer of a digital oscilloscope, and the resistance of the hollow hydrogel suture was measured with a 100 MΩ digital oscilloscope (from Tektronix). The hollow hydrogel suture was connected to the finger joint and knee joint, and connected to the digital oscilloscope to realize human motion perception.

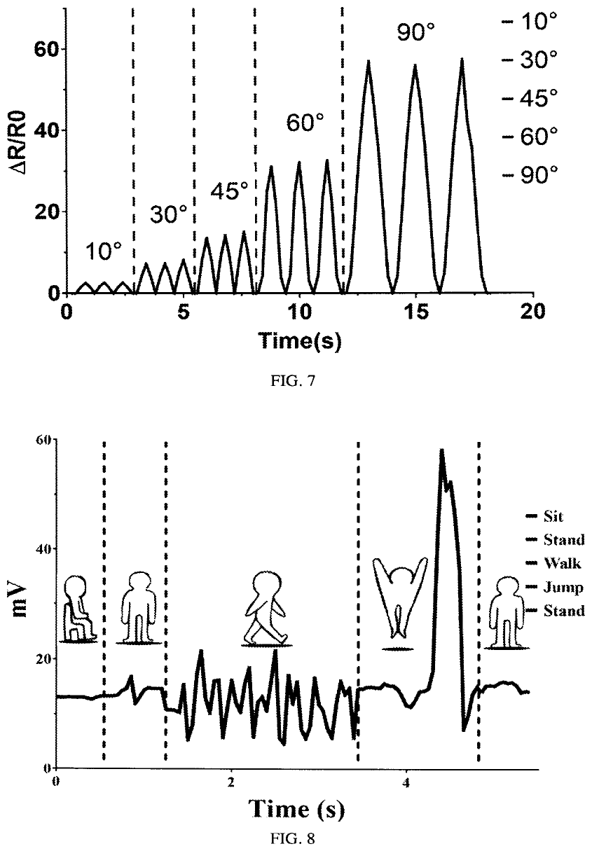

FIG. 7 shows test results of sensing performance (resistance change) of a hollow hydrogel suture for bidirectional liquid delivery and sensing communication (DTMS) according to an embodiment of the present disclosure. FIG. 8 shows test results of sensing performance (voltage change) of a hollow hydrogel suture for bidirectional liquid delivery and sensing communication (DTMS) according to an embodiment of the present disclosure.

As can be seen from FIG. 7 and FIG. 8, different motion amplitudes result in different electrical signals displayed in the sensor, which indicates that the hollow hydrogel suture for the bidirectional liquid delivery and sensing communication according to an embodiment of the present disclosure could achieve effective motion sensing.

### EXAMPLE 2

### Use of a Hollow Hydrogel Suture for Bidirectional Liquid Delivery and Sensing Communication in Disease Diagnosis-Treatment-Monitoring.

### (1) Use of the Hollow Hydrogel Suture for Bidirectional Liquid Delivery and Sensing Communication in Suture and Disease Diagnosis Test of Rats:

A hollow hydrogel suture for bidirectional liquid delivery and sensing communication (i.e., conductive hydrogel suture) was prepared according to (2) of Example 1, which had an inner diameter of 80 μm, and an outer diameter of 100 μm, and then subjected to a test as follows:

(i) Rats (SD rats, male, 230-250 g) were anesthetized by isoflurane inhalation. Skeletal muscle severing was performed near the hip joint on the dorsal side of the left lower limb of the rats, with a depth of 0.3-0.5 cm and a length of 1 cm. Continuous suture was performed on severed skeletal muscle with the hollow hydrogel suture for bidirectional liquid delivery and sensing communication. A wireless digital oscilloscope module was connected with two ends of the conductive hydrogel suture, for monitoring skeletal muscle discharge at a normal state.

A soft paralysis model was constructed by severing the sciatic nerve, and skeletal muscle discharges in the paralysis mode (Enteroparalysis) were recorded.

(ii) Rats (SD rats, male, 230-250 g) were anesthetized by isoflurane inhalation. After cutting off the trachea and performing tracheal intubation, the rats were supported by a ventilator. The pericardium was opened, and continuous suture was performed on the surface of myocardium with hollow hydrogel suture for bidirectional liquid delivery and sensing communication. The pericardium, intercostal space, pectoralis major muscle and skin were closed, and a thread end of the hollow hydrogel suture for bidirectional liquid delivery and sensing communication was led out to the subcutaneous, and connected with a wireless oscilloscope module, with connecting sites being sealed by insulative PDMS. Electrophysiological signals on the myocardial surface were recorded by using wireless oscilloscope module.

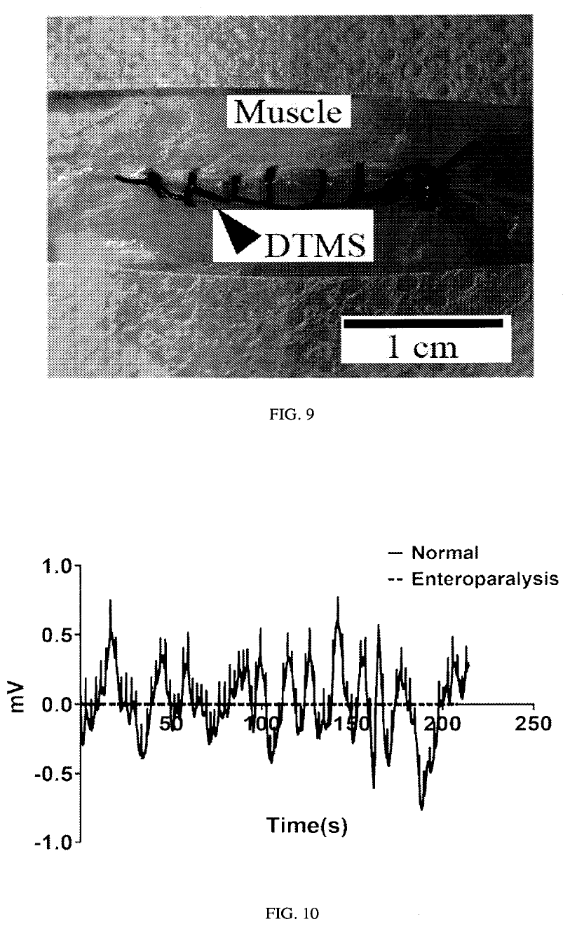

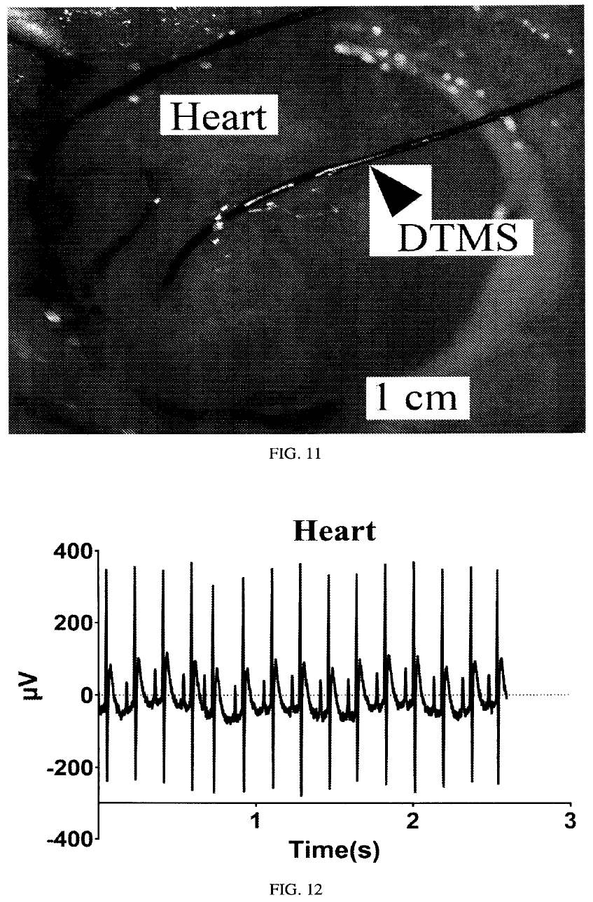

FIG. 9 shows result of muscle tissue sutured with a hollow hydrogel suture for bidirectional liquid delivery and sensing communication (DTMS) according to an embodiment of the present disclosure. FIG. 10 shows results of transmitting electromyography signals when suturing muscle tissue with a hollow hydrogel suture for bidirectional liquid delivery and sensing communication (DTMS) according to an embodiment of the present disclosure. FIG. 11 shows result of myocardial tissue sutured with a hollow hydrogel suture for bidirectional liquid delivery and sensing communication (DTMS) according to an embodiment of the present disclosure. FIG. 12 shows results of transmitting electrocardiogram signals when suturing cardiac muscle tissue with a hollow hydrogel suture for bidirectional liquid delivery and sensing communication (DTMS) according to an embodiment of the present disclosure.

As can be seen from FIGS. 10 and 12, DTMS could be used for suturing muscles and myocardium, and transmitting local electrophysiological signals from the tissue.

### (2) Perfusion Experiment of a Hollow Hydrogel Suture for Bidirectional Liquid Delivery and Sensing Communication on a Myocardium Surface of Rat(s).

A hollow hydrogel suture for bidirectional liquid delivery and sensing communication was prepared according to step (2) of Example 1, which had an inner diameter of 80 μm and an outer diameter of 100 μm, and then subjected to the test as follows:

Rats (SD rats, male, 230-250 g) were anesthetized by isoflurane inhalation. After cutting off the trachea and performing tracheal intubation, the rats were supported by a ventilator with mechanical ventilation; the left chest cavities of the rats were opened layer by layer, and the pericardiums of the rats were exposed. The pericardiums of the rats were opened, and continuous suture was then performed on the surface of the myocardium of each rat with the hollow hydrogel suture for bidirectional liquid delivery and sensing communication. For each rat, one end of the hollow hydrogel suture was led out to the subcutaneous area while the other end thereof was remained in the chest cavity. For each rat, the pericardium, intercostal space, pectoralis major muscle, and skin were closed, and the end of the hollow conductive hydrogel suture led out to the subcutaneous area was connected with the micro injection pump catheter. Near-infrared fluorescent dye Dir was perfused in a certain amount onto the myocardium surface of rats using a micro injection pump. A live fluorescence imaging was performed to observe the distribution, diffusion, and retention time of fluorescence on the myocardium surface of rats.

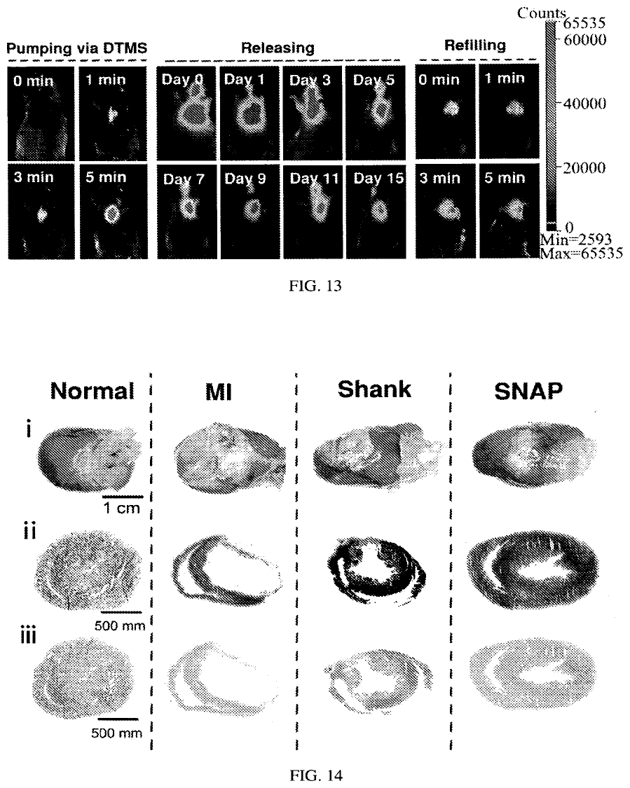

FIG. 13 shows results of near-infrared fluorescence imaging drugs delivered on myocardium surface of rats by a hollow hydrogel suture for bidirectional liquid delivery and sensing communication. As can be seen from FIG. 13, the hollow hydrogel suture according to the present disclosure could successfully deliver drugs into deep organs and tissues.

### (3) Therapeutic Effect of a Hollow Hydrogel Suture for Bidirectional Liquid Delivery and Sensing Communication on Myocardial Infarction in Rats.

A hollow hydrogel suture for bidirectional liquid delivery and sensing communication (i.e., conductive hydrogel suture) was prepared according to step (2) of Example 1, which had an inner diameter of 80 μm and an outer diameter of 100 μm, and then subjected to a test as follows:

Rats (SD rats, male, 230-250 g) were anesthetized by isoflurane inhalation. The rats were randomly divided into MI group, Shank group, and SNAP group, and Normal group as a control (i.e., Normal). After cutting off the trachea and performing tracheal intubation, the rats were supported by a ventilator with mechanical ventilation; the left chest cavities of the rats were opened layer by layer, and the pericardiums of the rats were exposed. The pericardiums of the rats were opened, and the left anterior descending coronary arteries (LAD) in the rats were ligated. Continuous suture was performed on the surface of infarcted myocardium in the ligated area of each rat with the hollow hydrogel suture for bidirectional liquid delivery and sensing communication. For each rat, one end of the the hollow hydrogel suture was extended to under the skin on the back and connected to an insulin pump reservoir. After suture implantation in each rat, the wound was sutured and sterilized, and 300 μL of physiological saline from a reservoir was injected into the rat of Shank group, while 300 μL 10 μmol/L SNAP solution from a reservoir was injected into the rat of SNAP group, but MI group was exposed for the same time after opening chest and myocardial infarction, followed by closure of the chest cavity without additional treatment. For each rat, the pericardium, intercostal space, pectoralis major muscle, and skin were closed, and penicillin sodium was intramuscularly injected once per day, which was maintained for 3 days. After 30 days, the rats were anesthetized and euthanized. After perfusion, the myocardium tissue was fixed and frozen sectioned, followed by staining with HE and Masson, and photographing. The staining results were statistically analyzed.

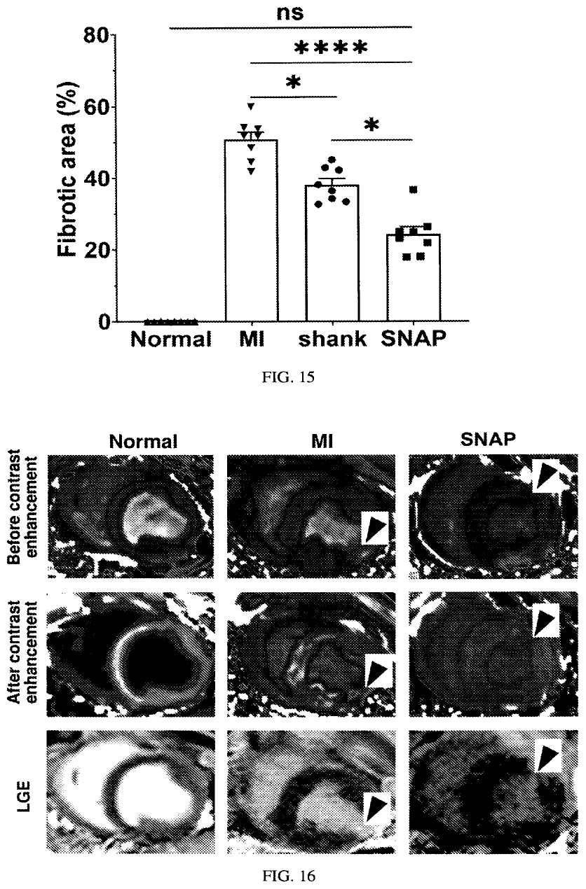

FIG. 14 shows histological staining results after improving myocardial infarction in rats with a hollow hydrogel suture for bidirectional liquid delivery and sensing communication. FIG. 15 shows statistical analysis of histological staining results after improving myocardial infarction in rats with a hollow hydrogel suture for bidirectional liquid delivery and sensing communication. As can be seen from FIGS. 14 to 15, using the hollow hydrogel suture for the bidirectional liquid delivery and sensing communication according to the present disclosure, therapeutic drugs could be pumped into the pathological tissue, thus effectively improving the myocardial infarction in rats.

### (4) Therapeutic Effect of a Hollow Hydrogel Suture for Bidirectional Liquid Delivery and Sensing Communication on Myocardial Infarction in Mini Pigs.

A hollow hydrogel suture for bidirectional liquid delivery and sensing communication (i.e., conductive hydrogel suture) was prepared according to step (2) of Example 1, which had an inner diameter of 950 μm, and an outer diameter of 1 mm, and then subjected to a test as follows:

Male mini pigs (1 year old, 30-40 kg) were randomly divided into Normal group, MI group, and SNAP group. All mini pigs were kept separately for one month and monitored daily. Before surgery, 10 mg·kg⁻¹ ketamine hydrochloride and 1 mg·kg⁻¹ rapid sleep anesthesia were injected into each of mini pigs. After inducing anesthesia. the mini pigs were fixed in a supine gesture on operating tables, and intubated for ventilation through the trachea, and the delivery of 2% isoflurane was maintained for anesthesia. After transferring the mini pigs to the right lateral position, thoracotomy was performed in a space between the fourth rib and the fifth rib. After using a rib opener to expand the surgical space, the pericardium of each mini pig was opened and 5-0 sutures were hung to observe LAD. The distal end of LAD was sealed with a 5-0 polypropylene suture to the second diagonal branch for 10 minutes, the surgical location was determined, and a pig acute myocardial infarction model was constructed. Electrocardiogram, respiration, body temperature, and blood oxygen saturation of each pig were monitored during the entire surgery. Intraoperative ST segment elevation serves as an indicator of acute myocardial infarction.

After the infarct area was visually determined, continuous suture was performed on the infarct area with the hollow hydrogel suture having an outer diameter of 1 mm according to the ischemic whitening area. The suture area did not exceed the ischemic area. The suture endpoints were introduced into the skin, and connected to a bluetooth ECG module and an insulin pump module respectively. After closing layer by layer, a mobile phone was used to monitor myocardial electrophysiology, and 1 mL 10 μmol/L SNAP solution was injected into the micro channel of the hollow hydrogel suture through a pump. Antibiotics were injected into the muscles every day after surgery to restore health.

After 30 days, all groups of mini pigs underwent myocardial magnetic resonance examination after anesthesia to evaluate myocardial infarction area and the therapeutic effect of the hollow hydrogel suture for bidirectional liquid delivery and sensing communication on mini pigs.

FIG. 16 shows results of cardiac magnetic resonance examination after improving myocardial infarction of mini pigs by a hollow hydrogel suture for bidirectional liquid delivery and sensing communication according to an embodiment of the disclosure. As can be seen from FIG. 16, magnetic resonance imaging shows a significant reduction in the myocardial infarction areas, and a significant recovery in the anterior ventricular wall thickness during systole and diastole.

### EXAMPLE 3

A mechanoluminescence hydrogel suture was prepared by Method II.

Preparation of a PVA solution: PVA was dissolved in deionized water at a mass fraction of 10%, and a resulting mixture was heated at 90° C. and stirred for 12 hours, obtaining a colorless and clear PVA solution.

Preparation of a mixed solution of PVA and copper-doped zinc sulfide (Cu:ZnS):Cu:ZnS was added to the PVA solution, such that a mass of Cu:ZnS accounted for 10% of the mass of PVA, and a resulting mixture was heated and stirred at 90° C. for 12 hours, obtaining the mixed solution of PVA and Cu:ZnS.

A glass fiber mold was cleaned with deionized water, and dried. The PVA solution was applied onto a surface of a pure aluminum wire mold. The pure aluminum wire mold coated with the PVA solution was dried overnight at room temperature, transferred to an oven at 60° C., and dehydrated and dried therein for 12 hours. The applying and the drying were repeated twice. A mixed solution of PVA and Cu:ZnS was applied onto a surface of polyvinyl alcohol coating after drying and dehydrating, dried overnight at room temperature, transferred to an oven at 60° C., and dehydrated and dried therein for 12 hours. The applying and the drying were repeated twice. A PVA solution was applied uniformly onto a surface of a PVA-Cu:ZnS coating after drying and dehydrating, dried overnight at room temperature, transferred to an oven at 60° C., and dehydrated and dried therein for 12 hours. The applying and the drying were repeated 5 times. The dried hydrogel suture and the mold were soaked in 6 mol/L NaOH solution and left to stand for 24 h. The hydrogel suture was transferred into deionized water, repeatedly shaken and rinsed for 48 h. The mold was removed from the hydrogel suture, obtaining a hollow hydrogel suture (i.e., mechanoluminescence hydrogel suture) for bidirectional liquid delivery and sensing communication.

Based on the use of pure aluminum wire molds having different diameters, a mechanoluminescence hydrogel suture having an inner diameter of 80 μm and an outer diameter of 100 μm, and a mechanoluminescence hydrogel suture having an inner diameter of 800 μm and an outer diameter of 1000 μm were obtained respectively.

After the obtained mechanoluminescence hydrogel sutures were freeze-dried, the hollow hydrogel sutures were adhered to a surface of a conductive adhesive substrate for gold spraying treatment; the hydrogel suture body and the hydrogel suture for bidirectional liquid delivery and sensing communication after the gold spraying treatment were characterized by scanning electron microscope (ZEISS).

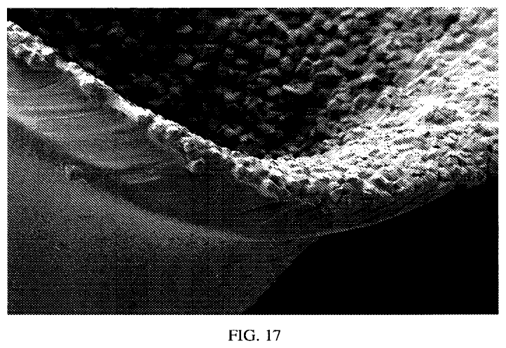

FIG. 17 shows a cross section of a mechanoluminescence hydrogel suture having an inner diameter of 80 μm, and an outer diameter of 100 μm. As can be seen from FIG. 17, the mechanoluminescence hydrogel suture has a smooth outer wall, while the inner wall thereof shows a rough particle-like structure due to the coating of copper-doped zinc sulfide crystals (the polyvinyl alcohol coating on the inner wall is thin, which is not obviously observed in the electron microscope image), indicating that polyethylene alcohol and copper-doped zinc sulfide have been successfully compounded in the hydrogel suture.

The prepared mechanoluminescence hydrogel suture (having an inner diameter of 800 μm, an outer diameter of 1000 μm, and a length of 2 cm) was cut down, fixed on a universal mechanical tester, and uniaxially stepping stretched at a frequency of 0.1 Hz and strain of 100%.

The results demonstrated that, the mechanoluminescence hydrogel suture emits strong light blue fluorescence under the uniaxial tension of the universal mechanical tester, indicating that the mechanoluminescence hydrogel suture has the function of responsive luminescence under the mechanical stimulation.

### EXAMPLE 4

A hollow hydrogel suture doped with both polypyrrole and copper-doped zinc sulfide was prepared according to Method II.

Preparation of a PVA solution: PVA was dissolved in deionized water at a mass fraction of 10%, and a resulting mixture was heated at 90° C. and stirred for 12 hours, obtaining a colorless and clear PVA solution.

Preparation of a PVA-PPy solution: PPy was added into the PVA solution, such that a mass of polypyrrole was 10% of a mass of PVA; a resulting mixture was heated and stirred at 90° C. for 12 hours until the solution was completely uniform and turn black. Preparation of a mixed solution of PVA and Cu:ZnS:

Cu:ZnS was added to the PVA solution, such that a mass of Cu:ZnS was 10% of a mass of PVA, and a resulting mixture was heated and stirred at 90° C. for 12 hours, obtaining the mixed solution of PVA and Cu:ZnS.

A glass fiber mold was washed with deionized water, and dried. The PVA solution was applied onto a surface of a pure aluminum wire mold. The pure aluminum wire mold coated with the PVA solution was dried overnight at room temperature, transferred to an oven at 60° C., and dehydrated and dried therein for 12 hours. The applying and the drying were repeated 5 times. The mixed hydrogel solution of PVA and PPy was uniformly applied onto a surface of polyvinyl alcohol coating after drying and dehydrating, dried overnight at room temperature, transferred to an oven at 60° C., and dehydrated and dried therein for 12 hours. The applying and the drying were repeated twice. The mixed solution of PVA and Cu: ZnS was applied uniformly onto a surface of a PVA-PPy coating after drying and dehydrating, dried overnight at room temperature, transferred to an oven at 60° C., and dehydrated and dried therein for 12 hours. The applying and the drying were repeated twice. The PVA solution was uniformly applied onto a surface of the PVA-Cu: ZnS coating after drying and dehydrating, dried at room temperature overnight, transferred to an oven at 60° C., and dehydrated and dried therein for 12 h. The applying and the drying were repeated twice. The dried hydrogel suture and the mold were soaked in 6 mol/L NaOH solution and left to stand for 24 h. The hydrogel suture was then transferred into deionized water, repeatedly shaken and rinsed for 48 h. The mold was removed from the hydrogel suture, obtaining a hollow hydrogel suture doped with both polypyrrole and copper-doped zinc sulfide. The hydrogel suture has dual functions of conductivity and mechanoluminescence.

The results of the above examples show that the hollow hydrogel suture according to the present disclosure could be used for signal transmission and drug delivery. It is composed of tough conductive hydrogel and a micro channel, and could realize effective sensing, monitoring, and evaluation of muscle movement, thrombosis, intestinal obstruction, and other scenes. In addition, the hollow hydrogel suture according to the

present disclosure could be used for myocardial infarction in rats. Its extremely low friction coefficient and mechanical properties matching with soft tissue would not damage myocardial tissue, but promotes ECG conduction, realizing real-time ECG mobile terminal monitoring. Also, the hollow hydrogel suture according to the present disclosure could deliver nitric oxide donor drugs sequentially according to demands, obviously inhibit inflammation, promote microvascular remodeling, and effectively improve cardiac function. Further, the hollow hydrogel suture according to the present disclosure realizes real-time sensing and controllable drug perfusion treatment in pigs after myocardial infarction. After one month of implantation in the myocardial infarction area, magnetic resonance imaging shows a significant reduction in the myocardial infarction area, and the anterior ventricular wall thickness during systole and diastole are significantly restored.

The above are only preferred embodiments of the present disclosure. It should be pointed out that for ordinary technicians in the art, several improvements and embellishments could be made without departing from the principles of the present disclosure, and these improvements and embellishments should also be deemed as falling within the scope of the present disclosure.

What is claimed is:

1. A hollow hydrogel suture for bidirectional liquid delivery and sensing communication, comprising
a hydrogel suture body, and
a compound with a sensing communication function, doped in the hydrogel suture body,
wherein the compound with the sensing communication function is at least one selected from the group consisting of polypyrrole and copper-doped zinc sulfide, and the hydrogel suture body is made of polyvinyl alcohol.

2. The hollow hydrogel suture for bidirectional liquid delivery and sensing communication as claimed in claim 1, wherein a mass fraction of the compound with the sensing communication function in the hollow hydrogel suture ranges from 1% to 30%.

3. The hollow hydrogel suture for bidirectional liquid delivery and sensing communication as claimed in claim 1, wherein the hollow hydrogel suture has an outer diameter of 80-1500 μm, an inner diameter of 50-1000 μm, and a length of 5-70 cm.

4. A method for preparing the hollow hydrogel suture for bidirectional liquid delivery and sensing communication as claimed in claim 1, comprising the steps of
(1) mixing polyvinyl alcohol, the compound with the sensing communication function, and water, to obtain a mixed solution;
(2) applying the mixed solution onto a surface of a line shape mold, and drying, to form a coating on the surface of the line shape mold; and
(3) soaking the line shape mold with the surface covered by the coating in a sodium hydroxide solution, hydrating, and removing the line shape mold, to obtain the hollow hydrogel suture for bidirectional liquid delivery and sensing communication.

5. The method as claimed in claim 4, wherein based on a total mass of the compound with the sensing communication function and polyvinyl alcohol being 100%, a mass fraction of the polyvinyl alcohol is not less than 70%.

6. The method as claimed in claim 4, wherein the polyvinyl alcohol has a weight average molecular weight of 7.5-50 kD.

7. A method for preparing the hollow hydrogel suture for bidirectional liquid delivery and sensing communication as claimed in claim 1, comprising the steps of
(i) applying a first polyvinyl alcohol solution onto a surface of a line shape mold and drying, to form a first polyvinyl alcohol coating on the surface of the line shape mold;
(ii) applying a mixed solution of polyvinyl alcohol and polypyrrole or a mixed solution of polyvinyl alcohol and copper-doped zinc sulfide onto a surface of the first polyvinyl alcohol coating, and drying, to form a sensing coating on the surface of the first polyvinyl alcohol coating;
(iii) applying a second polyvinyl alcohol solution onto a surface of the sensing coating, and drying, to form a second polyvinyl alcohol coating, thereby to obtain the line shape mold coated with a composite coating; and
(iv) soaking the line shape mold coated with the composite coating in a sodium hydroxide solution, hydrating, and removing the line shape mold, to obtain the hollow hydrogel suture for bidirectional liquid delivery and sensing communication.

8. The method as claimed in claim 7, wherein the first polyvinyl alcohol solution in step (i) and the second polyvinyl alcohol solution in step (iii) each have a polyvinyl alcohol mass fraction of 10-50%.

9. The method as claimed in claim 7, wherein the applying and the drying in step (i) are performed for 1-5 times, and under the condition that the applying and the drying in step
(i) are performed for more than once, the applying and the drying are alternately performed;
the applying and the drying in step (ii) are performed for 1-5 times, and under the condition that the applying and the drying in step (ii) are performed for more than once, the applying and the drying are alternately performed; and
the applying and the drying in step (iii) are performed for 1-5 times, and under the condition that the applying and the drying in step (iii) are performed for more than once, the applying and the drying are alternately performed.

10. The method as claimed in claim 7, wherein under the condition that the compound with sensing function includes both polypyrrole and copper-doped zinc sulfide, the step
(ii) comprises
applying a mixed solution of polyvinyl alcohol and copper-doped zinc sulfide onto a surface of the first polyvinyl alcohol coating, and drying, to form a polyvinyl alcohol coating doped with copper-doped zinc sulfide on the surface of the first polyvinyl alcohol coating; and applying a mixed solution of polyvinyl alcohol and polypyrrole onto a surface of the polyvinyl alcohol coating doped with copper-doped zinc sulfide, and drying, to form a polyvinyl alcohol coating doped with polypyrrole on the surface of the polyvinyl alcohol coating doped with copper-doped zinc sulfide; or
applying a mixed solution of polyvinyl alcohol and polypyrrole onto a surface of the first polyvinyl alcohol coating, and drying, to form a polyvinyl alcohol coating doped with polypyrrole on the surface of the first polyvinyl alcohol coating; and applying a mixed solution of polyvinyl alcohol and copper-doped zinc sulfide onto a surface of the polyvinyl alcohol coating doped with polypyrrole, and drying, to form a polyvinyl alcohol coating doped with copper-doped zinc sulfide on the surface of the polyvinyl alcohol coating doped with polypyrrole.

11. The method as claimed in claim 4, wherein a mass fraction of the compound with the sensing communication function in the hollow hydrogel suture ranges from 1% to 30%.

12. The method as claimed in claim 4, wherein the hollow hydrogel suture has an outer diameter of 80-1500 μm, an inner diameter of 50-1000 μm, and a length of 5-70 cm.

13. The method as claimed in claim 7, wherein a total mass fraction of the polyvinyl alcohol and the copper/zinc sulfide in the hollow hydrogel suture ranges from 1% to 30%.

14. The method as claimed in claim 7, wherein the hollow hydrogel suture has an outer diameter of 80-1500 μm, an inner diameter of 50-1000 μm, and a length of 5-70 cm.
